(19) United States
(12) Patent Application Publication
(10) Pub. No.: US 2012/0259366 A1
(43) Pub. Date: Oct. 11, 2012

(54) LUMBAR-SACRAL IMPLANT

(75) Inventor: Eric C. Lange, Pleasanton, CA (US)

(73) Assignee: Kyphon SARL., Neuchatel (CH)

(21) Appl. No.: 13/082,952

(22) Filed: Apr. 8, 2011

(51) Int. Cl. A61B 17/70; A61B 17/88

(52) U.S. Cl. 606/248; 606/279

(57) ABSTRACT

Medical devices for the treatment of spinal conditions are described herein. The medical device includes a main body that is adapted to be placed between the L5 vertebra and the sacrum so that the main body acts as a spacer with respect to the L5 vertebra and the sacrum to maintain distraction therebetween when the spine moves in extension. The main body is formed from two pieces, an upper body portion and a lower body portion.

### BACKGROUND

This invention relates generally to devices for the treatment of spinal conditions, and more particularly, to the treatment of various spinal conditions that cause back pain. Even more particularly, this invention relates to devices that may be placed between adjacent spinous processes to treat various spinal conditions. For example, spinal conditions that may be treated with these devices may include spinal stenosis, degenerative disc disease (DDD), disc herniations and spinal instability, among others.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis and other spinal conditions. The principal goal of surgery to treat lumbar spinal stenosis is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia and an incision is made in the patient to access the spine. The lamina of one or more vertebrae may be partially or completely removed to create more space for the nerves. The success rate of decompressive laminectomy has been reported to be in excess of 65%. A significant reduction of the symptoms of lumbar spinal stenosis is also achieved in many of these cases.

The failures associated with a decompressive laminectomy may be related to postoperative iatrogenic spinal instability. To limit the effect of iatrogenic instability, fixation and fusion may also be performed in association with the decompression. In such a case, the intervertebral disc may be removed, and the adjacent vertebrae may be fused. A discectomy may also be performed to treat DDD and disc herniations. In such a case, a spinal fusion would be required to treat the resulting vertebral instability. Spinal fusion is also traditionally accepted as the standard surgical treatment for lumbar instability. However, spinal fusion sacrifices normal spinal motion and may result in increased surgical complications. It is also believed that fusion to treat various spinal conditions may increase the biomechanical stresses imposed on the adjacent segments. The resultant altered kinematics at the adjacent segments may lead to accelerated degeneration of these segments.

As an alternative or complement to the surgical treatments described above, an interspinous process device may be implanted between adjacent spinous processes of adjacent vertebrae. The purposes of these devices are to provide stabilization after decompression, to restore foraminal height, and to unload the facet joints. They also allow for the preservation of a range of motion in the adjacent vertebral segments, thus avoiding or limiting possible overloading and early degeneration of the adjacent segments as induced by fusion. The vertebrae may or may not be distracted before the device is implanted therebetween. An example of such a device is the interspinous prosthesis described in U.S. Pat. No. 6,626,944, the entire contents of which are expressly incorporated herein by reference. This device, commercially known as the DIAM® spinal stabilization system, is designed to restabilize the vertebral segments as a result of various surgical procedures or as a treatment of various spinal conditions. It limits extension and may act as a shock absorber, since it provides compressibility between the adjacent vertebrae, to decrease intradiscal pressure and reduce abnormal segmental motion and alignment. This device provides stability in all directions and maintains the desired separation between the vertebral segments all while allowing motion in the treated segment.

Although currently available interspinous process devices typically work for their intended purposes, they could be improved. For example, where the spacer portion of the implant is formed from a hard material to maintain distraction between adjacent vertebrae, point loading of the spinous process can occur due to the high concentration of stresses at the point where the hard material of the spacer contacts the spinous process. This may result in excessive subsidence of the spacer into the spinous process. In addition, if the spinous process is osteoporotic, there is a risk that the spinous process could fracture when the spine is in extension. In addition, because of the human anatomy and the complex biomechanics of the spine, some currently available interspinous process devices may not be easily implantable in certain locations in the spine.

The spine is divided into regions that include the cervical, thoracic, lumbar, and sacrococcygeal regions. The cervical region includes the top seven vertebrae identified as C1-C7. The thoracic region includes the next twelve vertebrae identified as T1-T12. The lumbar region includes five vertebrae L1-L5. The sacrococcygeal region includes five fused vertebrae comprising the sacrum. These five fused vertebrae are identified as the S1-S5 vertebrae. Four or five rudimentary members form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebra angled from about 30 degrees to about 60 degrees in the horizontal plane. The S1 vertebra includes a spinous process aligned along a ridge called the medial sacral crest. However, the spinous process on the S1 vertebrae may not be well defined, or may be non-existent, and therefore may not be adequate for supporting an interspinous process device positioned between the L5 and S1 spinous processes.

Thus, a need exists for an interspinous process device that may be readily positioned between the L5 and S1 spinous processes. Moreover, there is a need to provide an interspinous process device that can provide dynamic stabilization to the instrumented motion segment and not affect adjacent segment kinematics.

### SUMMARY

A spinal implant is described herein that is particularly adapted for placement between the spinous processes of the L5 vertebra and the S1 vertebra to provide dynamic stabilization. The implant includes an upper saddle defined by a pair of sidewalls joined by a bottom wall. The upper saddle sidewalls may flare slightly outwardly away from the sagittal plane toward the top of the implant while the upper saddle bottom wall of the saddle may be concavely curved. In addition, the surfaces forming the upper saddle sidewalls and the upper saddle bottom wall extend in a direction, from the front of the implant to the rear of the implant, which is generally parallel to the sagittal plane. The upper saddle is configured to receive and support the spinous process of the L5 vertebra therein. The implant also includes a lower saddle defined by a pair of sidewalls joined by a top wall. The lower saddle sidewalls flare outwardly away from the sagittal plane toward the bottom of the implant. In addition, the surfaces forming the lower saddle sidewalls extend in a direction, from the front of the implant to the rear of the implant, outwardly away from the sagittal plane. The lower saddle top wall may be concavely curved. In addition, the surface forming the lower saddle top wall extends in a direction, from the front of the implant to the rear of the implant, toward the top of the implant. The lower saddle is not intended to engage and is not supported by the spinous process of the S1 vertebra. Rather the lower saddle merely provides a space into which that spinous process may extend when the implant is properly located in place.

The spinal implant described herein has outer sidewalls that extend on either side of the implant from the upper portion of the implant to the lower portion of the implant. The outer sidewalls flare outwardly away from the sagittal plane from the upper portion of the implant to give the implant a generally triangular-like shape. The wider bottom portion of the implant allows two lower lobes to be defined along the bottom portion of the implant adjacent to either side of the lower saddle. The lower lobes each define a channel extending through the thickness of the implant. The channels allow a fixation device to extend therethrough to fix the implant in the desired location. For example, screws may be used to extend through the channels such that they would engage the pedicles of the S1 vertebra. The channels flare outwardly from adjacent to the top of the bottom portion of the implant around the midline. For example, the longitudinal axes of the channels extend at an angle of about 60 degrees away from the sagittal plane toward the rear of the implant and at an angle of about 5 degrees toward the top of the implant in a direction from the front of the implant toward the rear of the implant.

The spinal implant is formed from two portions. An inferior portion and a superior portion. The inferior portion may be made from a solid or relatively stiff material such as PEEK, a high durometer polycarbonate-urethane (“PCU”), stainless steel, titanium or other hard, durable biocompatible material. By forming the inferior portion from a relatively stiff material, the fixation device can firmly affix the inferior portion of the spinal implant to the spine while ensuring that the inferior portion will not be pulled from the fixation device during flexion or other movement of the spine. Such pulling through of the fixation device from the implant is more likely if the inferior portion were formed from a flexible material. Conversely, the superior portion may be formed from a softer more flexible material, such as silicone, or a low durometer PCU or some other flexible biocompatible material. Forming the superior portion from a flexible material prevents subsidence, which may occur when the superior spinous process engages a hard material such as metal. In addition, forming the superior portion from a flexible material provides adequate stabilization to the L5/S1 level. More importantly, forming the superior portion from a flexible material allows the implant to act as a shock absorber in extension while providing adequate stabilization to the L5/S1 level and allow for a more normal range of motion. Appropriate connection means may be used to connect the inferior portion of the spinal implant to the superior portion of the spinal implant.

### BRIEF DESCRIPTION OF THE DRAWINGS

FIG. 1 is a front perspective view of one embodiment of a lumbar-sacral implant with the superior portion separated from the inferior portion;

FIG. 1A is a front perspective view of another embodiment of a lumbar-sacral implant with the superior portion separated from the inferior portion;

FIG. 2 is a rear perspective view of the embodiment of a lumbar-sacral implant shown in FIG. 1 but with the superior portion connected to the inferior portion;

FIG. 3 is a bottom perspective view of the embodiment of a lumbar-sacral implant shown in FIG. 2;

FIG. 4 is a rear elevation view of the embodiment of a lumbar-sacral implant shown in FIG. 2;

FIG. 5 is a cross-sectional view of the embodiment of a lumbar-sacral implant shown in FIG. 2 taken along line V-V in FIG. 3;

FIG. 6 is a schematic view of the cross-section view of the embodiment of a lumbar-sacral implant shown in FIG. 5 located between the L5 spinous process and the sacrum;

FIG. 7 is a cross-sectional view of the embodiment of a lumbar-sacral implant shown in FIG. 2 taken along line VII-VII FIG. 3;

FIG. 8 is a side elevation view of the lumbar-sacral implant shown in FIG. 2;

FIG. 9 is a front elevation view of the lumbar-sacral implant shown in FIG. 2 mounted on a spine; and

FIG. 10 is a side elevation view of the lumbar-sacral implant shown in FIG. 2 mounted on a spine.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

As used in this specification and the appended claims, the terms “upper”, “top”, “lower”, “bottom”, “front”, “back”, “rear”, “left”, “right”, “side”, “middle” and “center” refer to portions of or positions on the implant when the implant is oriented in its implanted position.

As used in this specification and the appended claims, the term “axial plane” when used in connection with particular relationships between various parts of the implant means a plane that divides the implant into upper and lower parts. As shown in the FIGS., the axial plane is defined by the X axis and the Z axis. As used in this specification and the appended claims, the term “coronal plane” when used in connection with particular relationships between various parts of the implant means a plane that divides the implant into front and back parts. As shown in the FIGS., the coronal plane is defined by the X axis and the Y axis. As used in this specification and the appended claims, the term “sagittal plane” when used in connection with particular relationships between various parts of the implant means a plane that divides the implant into left and right parts. As shown in the FIGS., the sagittal plane is defined by the Y axis and the Z axis.

As used in this specification and the appended claims, the term “body” when used in connection with the location where the device of this invention is to be placed to treat spinal disorders, or to teach or practice implantation methods for the device, means a mammalian body. For example, a body can be a patient's body, or a cadaver, or a portion of a patient's body or a portion of a cadaver.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, “perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

A spinal implant 10 is described herein that is particularly adapted for placement between the spinous processes of the L5 vertebra and the S1 vertebra. However, it is to be understood that even though the following description of implant 10 is provided with reference to the L5 spinous process and the S1 spinous process, implant 10 may be used between other adjacent spinous processes and the discussion of the L5 spinous process may be interpreted to include any superior spinous process and the S1 spinous process may be interpreted to include the adjacent inferior spinous process.

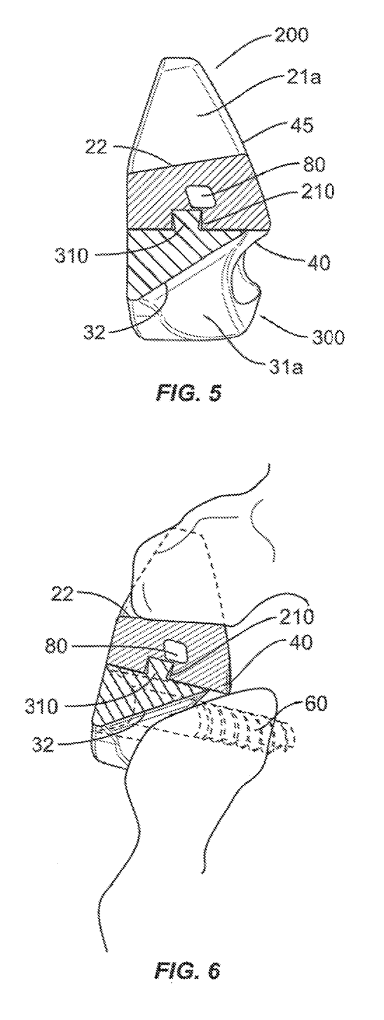

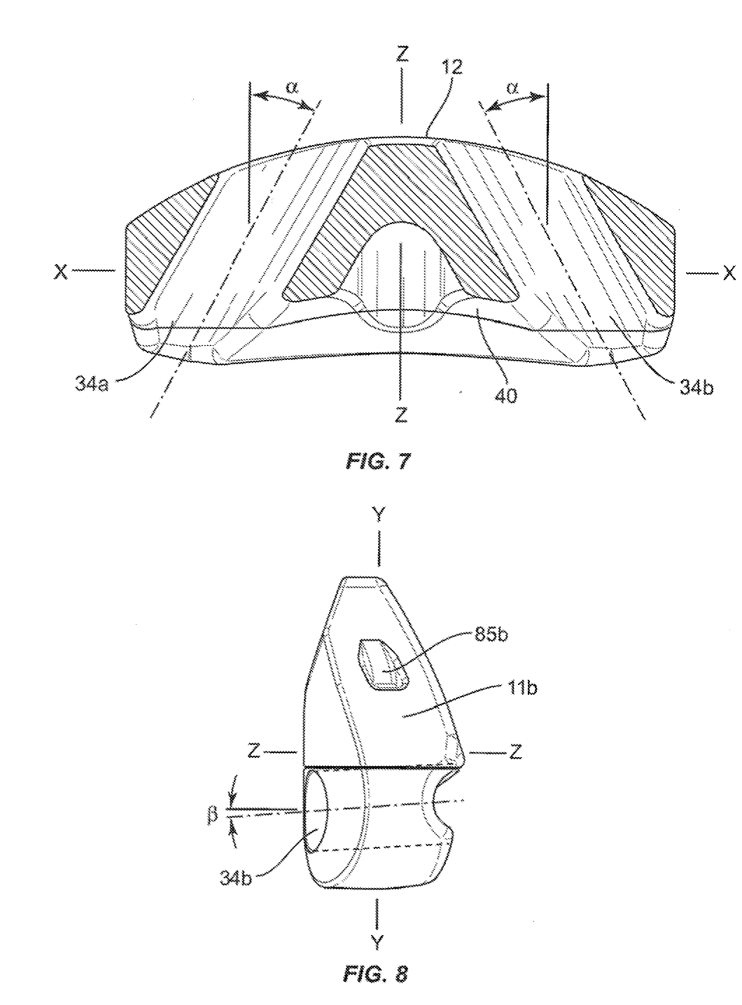

Implant 10 includes an upper saddle 20 defined by a pair of sidewalls 21a and 21b joined by a bottom wall 22. Upper saddle sidewalls 21a and 21b may flare slightly outwardly away from the sagittal plane toward the top of implant 10 while upper saddle bottom wall 22 may be concavely curved. Implant 10 may have a variable radius, which may be from about 3.0 mm on the ventral face 12 to about 2.0 mm on the dorsal face 45. This allows implant 10 to engage the L5 spinous process, which is usually thicker at the base. As shown in FIG. 5, upper saddle bottom wall 22 may be oriented at about a 10 degree angle in the sagittal plane. The angle could be as large as about 20 degrees. The surfaces forming upper saddle sidewalls 21a and 21b and upper saddle bottom wall 22 may be generally parallel to the sagittal plane. This configuration for upper saddle 20 allows upper saddle 20 to receive and support the spinous process of an L5 vertebra therein. The height of upper saddle sidewalls 21a and 21b should be chosen so that upper saddle sidewalls 21a and 21b prevent the upper portion of implant 10 from moving laterally out of engagement with the spinous process of the L5 vertebra. Upper saddle sidewalls 21a and 21b may extend between ⅓ and ½ of the base of the spinous process so they engage the lamina by about 2 to 3 mm. Upper saddle sidewalls 21a and 21b may not have a constant cross-section. This allows upper saddle 20 to accommodate the variable thickness of the spinous process. Implant 10 also includes a lower saddle 30 defined by a pair of sidewalls 31a and 31b joined by a top wall 32. As described in more detail below, lower saddle 30 has a configuration to provide clearance of implant 10 over the S1 spinous process. As such, lower saddle 30 would not engage the spinous process of the S1 vertebra. Lower saddle sidewalls 31a and 31b flare outwardly away from the sagittal plane toward the bottom of implant 10.

Upper saddle sidewalls 21a and 21b flare out and may have a variable angle. The angle starts at about 40 degrees at the upper portion of upper saddle 20 and varies so that the angle is about 25 degrees at about the lowermost portion of upper saddle 20. Lower saddle sidewalls 31a and 31b flare out and have a constant angle between about 25 degrees and about 35 degrees. Lower saddle top wall 32 may be concavely curved or may have another configuration that allows the lower portion of implant 10 to be fixed to the S1 pedicles and minimize any interference between the S1 spinous process and the rear of implant 10. Lower saddle top wall 32 is inclined between about 30 degrees and about 35 degrees in the sagittal plane.

Implant 10 has outer sidewalls 11a and 11b that extend on either side of implant 10 from the upper portion of implant 10 to the lower portion of implant 10. Outer sidewalls 11a and 11b flare outwardly away from the sagittal plane from the upper portion of implant 10 to give implant 10 a generally triangular-like shape. In addition, the overall shape of implant 10 transfers load from the L5 spinous process to the S1 pedicles instead of to the S1 spinous process or the S1 laminae. This is especially helpful where implant 10 is used in the L5-S1 level since the small size and shape of the S1 spinous process may not provide adequate support for an implant.

The front face 12 of implant 10 may have a curved profile that tapers from about 0 degrees along the middle of front face 12 to about 35 degrees adjacent to sidewalls 11a, 11b. Implant 10 may have a curvature radius of between about 20 mm and about 30 mm. The generally triangular shape, where the base is larger than the top results in a constant pressure applied along the cross-sectional area of implant 10. The shape of implant 10 also provides a better fit in the L5/S1 space and therefore offers stability for implant 10. The rear of implant 10 has a stepped configuration and includes a shelf 40 separating the rear of implant 10 into an upper portion and a lower portion. Shelf 40 may be curved and is located so it is generally aligned with or above channels 34a and 34b. Shelf 40 acts as a transition between the upper and lower portions of the rear of implant 10 and ensures that implant 10 will fit properly in the patient's anatomy. The upper rear portion of implant 10 is defined by the rear wall 45, which flares outwardly from the top of implant 10. Rear wall 45 is curved such that it does not compete for engagement with upper saddle 20 but rather allows implant 10 to rest freely on the L5 lamina. This allows for easy implantation on the L5 level. The thickness of implant 10 gradually increases from the top of implant 10 to shelf 40. This taper may be between about 30 degrees and about 50 degrees. The bottom rear portion of implant 10 has a thinner profile and provides clearance so that lower saddle 30 does not engage the inferior spinous process. This results in practically no load being transferred from implant 10 to the inferior spinous process. Indeed, lower saddle 30 may be configured such that it is spaced from and does not engage the inferior spinous process when implant 10 is implanted in the patient.

The wider bottom portion of implant 10 allows two lower lobes 33a and 33b to be defined along the bottom portion of implant 10 adjacent to either side of lower saddle 30 and provides an area through which implant 10 may be fixed to the spine. Each lower lobe 33a and 33b defines a channel 34a and 34b extending through implant 10. Channels 34a and 34b allow a fixation device 60, such as a cortical screw or similar device, to extend therethrough to fix implant 10 in the desired location on the spine. As such, the internal diameter of channels 34a and 34b should be sufficient to allow passage of fixation device 60 therethrough, but should not be so large as to allow too much “play”, or too big of a gap, between fixation device 60 and channels 34a and 34b. For example, channels 34a and 34b could have an internal diameter that is about 0.5 mm to about 1 mm greater than the outer diameter of fixation device 60. Channels 34a and 34b flare outwardly from adjacent the mid-line of implant 10 and adjacent the top of the bottom portion of implant 10 so that fixation device 60 can be located therein and extend to the pedicles of the S1 vertebra. For example, channels 34a and 34b may extend at an angle α of about 60 degrees away from the sagittal plane toward the rear of implant 10 and at an angle β of about 5 degrees toward the top of implant 10 in a direction from the front of implant 10 toward the rear of implant 10. Alternatively, angle α could be between about 45 degrees and about 60 degrees, while angle β could be between about 5 degrees and about 10 degrees. This orientation for channels 34a and 34b allows fixation device 60 to extend there through and engage the pedicles of the S1 vertebra. The pedicles have good bone quality and provide superior support for spinal stabilization systems. The wider bottom portion of implant 10, and indeed the overall configuration of implant 10, also allows implant 10 to withstand higher forces being placed on it and helps to ensure compression forces placed on implant 10 are evenly distributed throughout the body of implant 10.

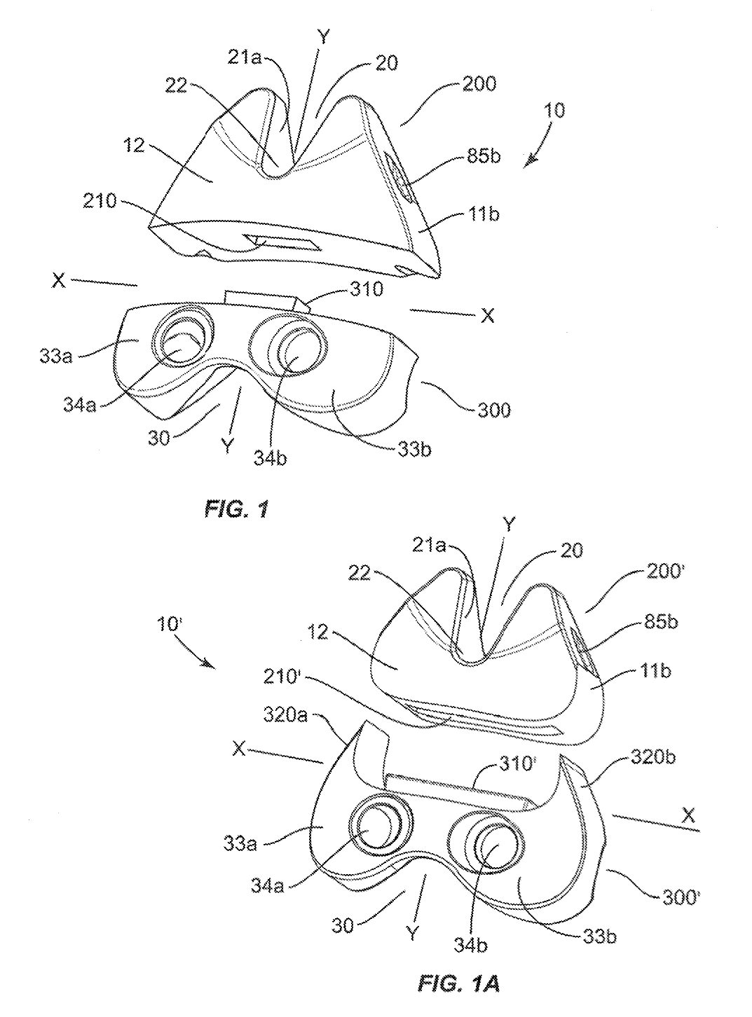

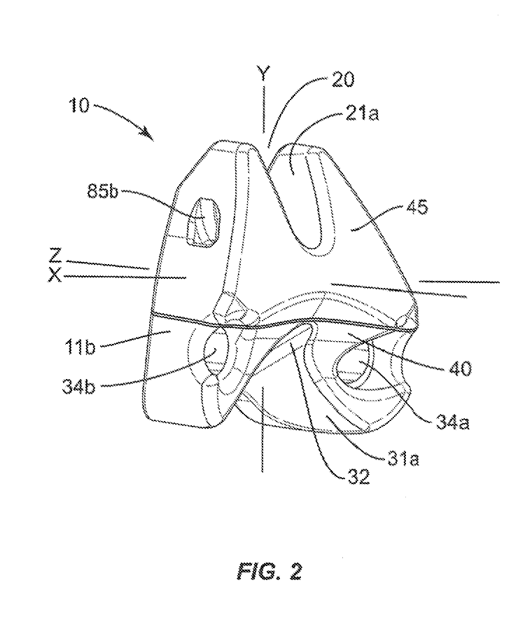

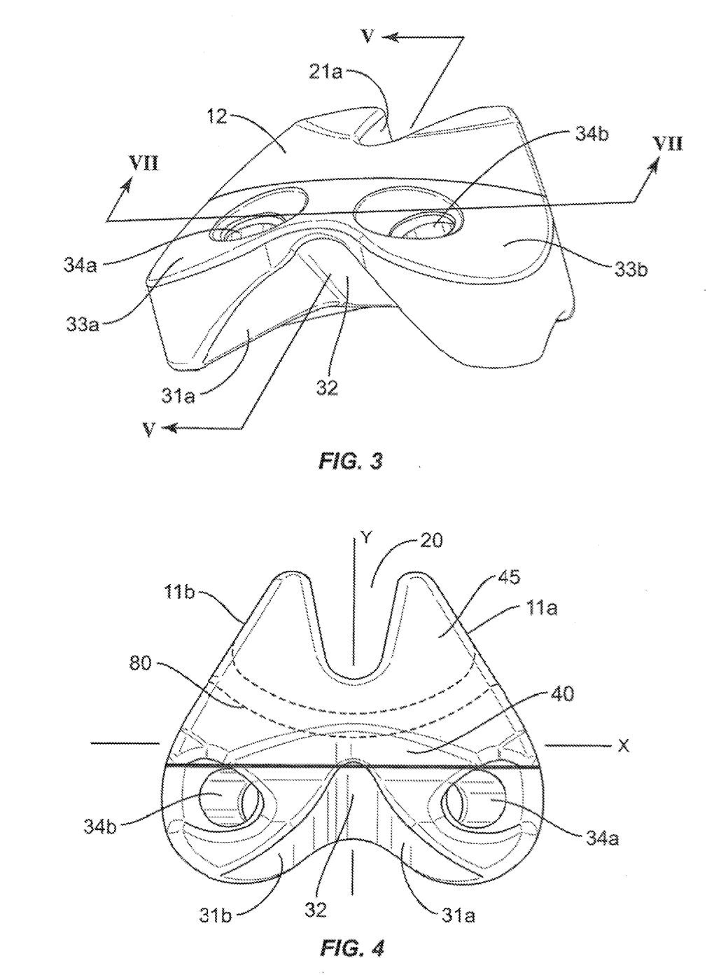

Implant 10 may be formed from two portions. An inferior portion 300 and a superior portion 200. Inferior portion 300 may be made from a solid or relatively stiff material such as PEEK, a high durometer polycarbonate-urethane (“PCU”), stainless steel, titanium or other hard, durable biocompatible material. By forming inferior portion 300 from a relatively stiff material, fixation device 60 can firmly affix inferior portion 300 to the spine while ensuring that inferior portion 300 will not be pulled from fixation device 60 during flexion or other movement of the spine. Such pulling through of a spinal implant from a fixation device is more likely if the implant were formed from a softer, more flexible material. Conversely, superior portion 200 may be formed from a softer more flexible material, such as silicone, a low durometer PCU or some other flexible biocompatible material. Superior portion 200 may have a durometer of between about 63A and about 85A. Forming superior portion 200 from a flexible material prevents subsidence, which may occur when the superior spinous process engages a hard material such as metal. More importantly, forming superior portion 200 from a flexible material allows implant to act as a shock absorber in extension while providing adequate stabilization to the L5/S1 level and allowing a more normal range of motion. As shown in FIG. 1, inferior portion 300 may be designed to extend only below, or inferior to, superior portion 200. In an alternate embodiment shown in FIG. 1A, inferior portion 300′ includes superiorly extending lateral portions 320a and 320b. This configuration provides implant 10 with a varying durometer laterally across implant 10 where the sides are stiffer than the central portion of implant 10.

Appropriate connection means may be used to connect inferior portion 300 to superior portion 200. For example, a tab 310 may extend from the upper wall 320 of inferior portion 300 which engages a slot 210 that may be formed in the bottom portion of superior portion 200, or vice versa. Tab 310 may have a generally elongated cross section when view from the top of inferior portion 300. As shown in FIG. 1, tab 310 may extend only along a portion of upper wall 320. Alternatively, as shown in FIG. 1a, tab 310′ may extend across substantially the entire width of upper wall 320′. The specific dimensions of the tab may be varied as necessary. In addition, the cross-section of the lower portion of tab 310 may be smaller than the cross-section of the upper portion of tab 310. See FIGS. 5 and 6. Slot 210 may be formed with a configuration and dimensions that will allow tab 310 to be received in slot 210 with an interference fit. The configuration for tab 310 and slot 210 ensures that inferior portion 300 is locked to superior portion 200 with no relative movement between them. In addition to the use of a single slot 210 and tab 310, other connection means may be used to connect inferior portion 300 to superior portion 200. For example, a tab in the form of a helical screw could engage a tapped hole, the tab could take the form of a barb, multiple slots and tabs could be used, appropriate adhesives could be used, a tongue and groove configuration could be used, or any other connection system known to those of skill in the art could be used. Another mechanism to connect inferior portion 300 to superior portion 200 is to overmold superior portion 200 over inferior portion 300.

An advantage of a two-piece implant as described herein, is that the inferior portion may be implanted and fixed in placed first and then the superior portion may be located between the inferior portion and the superior spinous process. Once the inferior portion is properly located in the interspinous space adjacent to the S1 vertebra, fixation devices, such as cortical screws, may be placed through channels 34a and 34b and driven into the S1 pedicles to fix the inferior portion in place. Thereafter, the superior portion may be fitted between the L5 vertebra and the inferior portion of the implant. This may make implantation of the implant easier than if the implant were a single piece. If desired, a tether 90, or other fixation device, may be used to connect the superior portion of the implant to the superior spinous process.

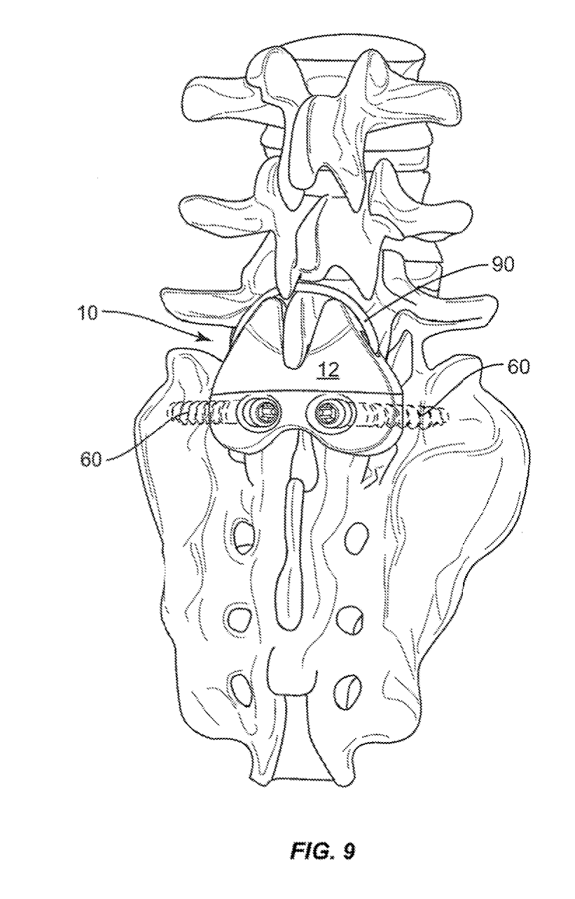

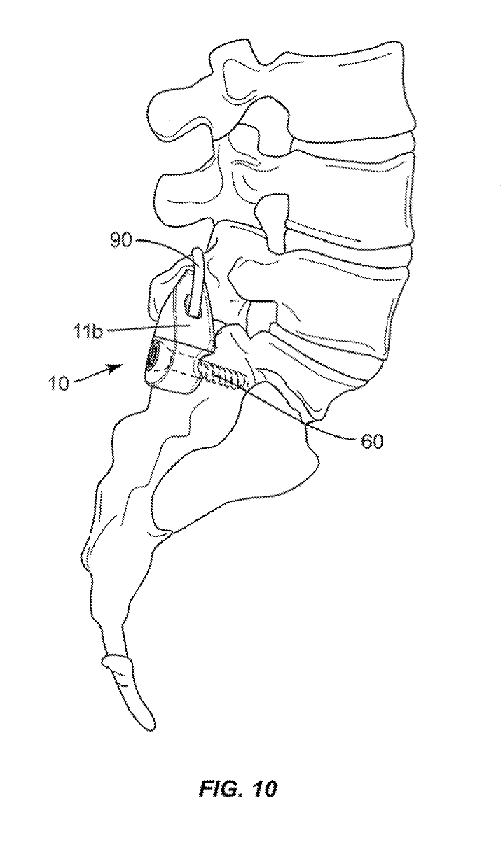

Implant 10 may also define a curved passage 80 that extends between outer sidewalls 11a and 11b of implant 10. The curve of passage 80 may be defined by a radius of curvature of about 20 millimeters where the openings 85a and 85b to passage 80 are closer to the top of implant 10 than the nadir of passage 80. Openings 85a and 85b are generally perpendicular to outer sidewalls 11a and 11b. Other radii of curvature may also be used to define passage 80. The nadir of passage 80 may be substantially aligned in the sagittal plane with the bottom most portion of upper saddle bottom wall 22 and the uppermost portion of lower saddle top wall 32. A tether 90 may extend through passage 80. The curve of passage 80 facilitates tether 90 being threaded through passage 80 with a standard curved surgical needle. As shown in FIGS. 9 and 10, tether 90 may extend across the superior portion of the superior spinous process when implant 10 is located in the interspinous space. Tether 90 thus helps to maintain implant 10 in the proper position in the patient's anatomy during extension and flexion. It is to be understood that other fixation devices may be used instead of a tether 90. For example, a pin, rod, screw or other similar mechanical device may be used and would extend through upper saddle 20 and into the upper spinous process.

While various embodiments of the flexible interspinous process device have been described above, it should be understood that they have been presented by way of example only, and not limitation. Many modifications and variations will be apparent to the practitioner skilled in the art. The foregoing description of the flexible interspinous process device is not intended to be exhaustive or to limit the scope of the invention. It is intended that the scope of the invention be defined by the following claims and their equivalents.

What is claimed is:

1. A device, comprising:
a front face;
a rear face;
an upper body portion defining an upper saddle wherein the upper body portion is formed from a flexible material;
a lower body portion defining a lower saddle; the upper body portion separated from the lower body portion by an axial plane wherein the lower body portion is formed from a material that is stiffer than the upper body portion;
the lower body portion including a left lower lobe and a right lower lobe, each lobe being adjacent to an opposite side of the lower saddle;
a left sidewall, a right sidewall and a sagittal plane dividing the device into a left part and a right part, the left sidewall and the right sidewall each extending from the upper body portion to the lower body portion and extending away from the sagittal plane in a direction from the upper body portion to the lower body portion such that a first distance between the left sidewall and the right sidewall adjacent to the upper body portion is less than a second distance between the left sidewall and the right sidewall adjacent to the lower body portion; and
a left channel extending through the device in the left lower lobe and a right channel extending through the device in the right lower lobe.

2. The device of claim 1 wherein the left channel extends from the front face adjacent to a midline and a superior portion of the lower body portion.

3. The device of claim 2 wherein the left channel is oriented at an angle of about 60 degrees away from the sagittal plane in a direction from the front face to the rear face.

4. The device of claim 1, 2 or 3 wherein the left channel is oriented at an angle of about 5 degrees toward the axial plane in a direction from the lower body portion to the upper body portion.

5. The device of claim 1 wherein the right channel extends from the front face adjacent to a midline and a superior portion of the lower body portion.

6. The device of claim 5 wherein the right channel is oriented at an angle of about 60 degrees away from the sagittal plane in a direction from the front face to the rear face.

7. The device of claim 1, 2, 3, 5 or 6 wherein the right channel is oriented at an angle of about 60 degrees away from the sagittal plane in a direction from the front face to the rear face.

8. The device of claim 4 wherein the right channel is oriented at an angle of about 60 degrees away from the sagittal plane in a direction from the front face to the rear face.

9. The device of claim 1, 2, 3, 5, 6, or 8 wherein the right channel is oriented at an angle of about 5 degrees toward the axial plane in a direction from the lower body portion to the upper body portion.

10. The device of claim 4 wherein the right channel is oriented at an angle of about 5 degrees toward the axial plane in a direction from the lower body portion to the upper body portion.

11. The device of claim 7 wherein the right channel is oriented at an angle of about 5 degrees toward the axial plane in a direction from the lower body portion to the upper body portion.

12. The device of claim 9 wherein the right channel is oriented at an angle of about 5 degrees toward the axial plane in a direction from the lower body portion to the upper body portion.

13. A method of implanting a device having
a front face;
a rear face;
an upper body portion defining an upper saddle wherein the upper body portion is formed from a flexible material;
a lower body portion defining a lower saddle; the upper body portion separated from the lower body portion by an axial plane wherein the lower body portion is formed from a material that is stiffer than the upper body portion;
the lower body portion including a left lower lobe and a right lower lobe, each lobe being adjacent to an opposite side of the lower saddle;
a left sidewall, a right sidewall and a sagittal plane dividing the device into a left part and a right part, the left sidewall and the right sidewall each extending from the upper body portion to the lower body portion and extending away from the sagittal plane in a direction from the upper body portion to the lower body portion such that a first distance between the left sidewall and the right sidewall adjacent to the upper body portion is less than a second distance between the left sidewall and the right sidewall adjacent to the lower body portion; and
a left channel extending through the device in the left lower lobe and a right channel extending through the device in the right lower lobe; comprising:
implanting the lower body portion into an interspinous space defined between an inferior spinous process and a superior spinous process;
placing a first fixation device through one of the left channel or the right channel and placing a second fixation device through the other of the right channel or the left channel; and
implanting the upper body portion between the lower body portion and the superior spinous process.

14. The method of claim 14 further comprising extending a tether from the upper body portion and around the superior spinous process.
